(19) United States
(12) Patent Application Publication
(10) Pub. No.: US 2013/0023788 A1
(43) Pub. Date: Jan. 24, 2013

(54) GASTROINTESTINAL BIOPSY DEVICES

(71) Applicants: Christopher J. Gostout, Rochester, MN (US); Kevin E. Bennet, Rochester, MN (US); Elizabeth Rajan, Rochester, MN (US); Eduardo Aimore Bonin, Rochester, MN (US)

(72) Inventors: Christopher J. Gostout, Rochester, MN (US); Kevin E. Bennet, Rochester, MN (US); Elizabeth Rajan, Rochester, MN (US); Eduardo Aimore Bonin, Rochester, MN (US)

(21) Appl. No.: 13/551,444

(22) Filed: Jul. 17, 2012

Related U.S. Application Data
(60) Provisional application No. 61/509,027, filed on Jul. 18, 2011.

(51) Int. Cl. A61B 10/04

(52) U.S. Cl. 600/564; 600/562

(57) ABSTRACT

This document provides methods and materials involved in obtaining biopsy material from the gastrointestinal tract of a mammal (e.g., a human). For example, devices (e.g., percutaneous biopsy devices) configured to allow a user (e.g., a surgeon) to obtain biopsy material from the gastrointestinal tract of a mammal in a minimally-invasive manner as well as methods for using biopsy devices are provided.

### CROSS REFERENCE TO RELATED APPLICATIONS

This application claims the benefit of U.S. Provisional Application Ser. No. 61/509,027, filed Jul. 18, 2011. The disclosure of the prior application is considered part of (and is incorporated by reference in) the disclosure of this application.

### BACKGROUND

1. Technical Field

This document relates to methods and materials involved in obtaining biopsy material from the gastrointestinal tract of a mammal (e.g., a human). For example, this document relates to devices (e.g., percutaneous biopsy devices) configured to allow a user (e.g., a surgeon) to obtain biopsy material from the gastrointestinal tract of a mammal in a minimally-invasive manner.

2. Background Information

Tissue samples are routinely removed from a living mammal to determine the presence or extent of a disease. In such cases, the tissue biopsy is typically examined under a microscope by a pathologist. There are several different types of biopsy procedures. For example, when an entire lump or suspicious area is removed, the procedure can be called an excisional biopsy. When a sample of tissue is removed with preservation of the histological architecture of the tissue's cells, the procedure can be called an incisional biopsy or core biopsy. When a sample of tissue or fluid is removed with a needle in such a way that cells are removed without preserving the histological architecture of the tissue cells, the procedure can be called a needle aspiration biopsy.

### SUMMARY

This document provides methods and materials involved in obtaining biopsy material from the gastrointestinal tract of a mammal (e.g., a human). For example, this document provides devices (e.g., percutaneous biopsy devices) configured to allow a user (e.g., a surgeon) to obtain biopsy material from the gastrointestinal tract of a mammal in a minimally-invasive manner as well as methods for using biopsy devices. In some cases, the methods and materials provided herein can be used in a medical setting to allow a surgeon to acquire large pieces of gastrointestinal tract tissue (e.g., stomach, small bowel, or colon tissue) safely. In some cases, the methods and materials provided herein can be used to obtain large core diameter samples of a particular selected depth (e.g., a depth selected to obtain desired tissue or tissue having a desired cell type). For example, the methods and materials provided herein can be used to obtain core samples having a depth such that the core includes serosa, muscle, submucosa, and/or mucosa. In some cases, the methods and materials provided herein can be used to obtain a tissue sample having enteric nerves, interstitial cells of Cajal (ICC), and/or immune cells. Having the ability to obtain desired tissue samples as described herein can allow pathologists to determine the presence or extent of a disease quickly and effectively.

In general, one aspect of this document features a system for obtaining a tissue biopsy from the gastrointestinal tract of a mammal. The system comprises, or consists essentially of, (a) a hollow member configured to be positioned near gastrointestinal tissue to be obtained, (b) a tissue collection element configured to be advanced within a lumen of the hollow member, wherein the tissue collection element defines a lumen configured to house at least a portion of the tissue biopsy, and (c) a positioning element configured to be advanced within the lumen of the tissue collection element or the lumen of the hollow member, wherein the positioning element is capable of being advanced into gastrointestinal tract tissue and capable of positioning the gastrointestinal tract tissue into the lumen of the tissue collection element upon retraction of the positioning element. The mammal can be a human. The hollow member can be a flexible catheter or a rigid catheter. The gastrointestinal tract tissue can be a polyp. The gastrointestinal tract tissue can be normal or abnormal. The tissue collection element can comprise one or more blades. The tissue collection element can define one or more ports configured to deliver material from outside of the body of the mammal to the gastrointestinal tract of the mammal. The hollow member can comprise a bumper element. The hollow member can comprise a magnetic alignment element. The system can comprise a snare. The positioning element can comprise a screw-like structure.

In another aspect, this document features a method for obtaining a tissue biopsy from a gastrointestinal tract of a mammal. The method comprises, or consists essentially of: (a) inserting a member having a distal end and a proximal end into the mammal to position the distal end of the member proximal to tissue of the gastrointestinal tract, wherein the member defines a lumen, (b) positioning a tissue collector element within the lumen of the member, wherein the tissue collector element defines a lumen configured to house the tissue biopsy, (c) advancing a positioning element having a distal end and a proximal end within the lumen of the member or the lumen of the tissue collector element, (d) inserting the positioning element into tissue of the gastrointestinal tract, (e) advancing the tissue collector element into the tissue of the gastrointestinal tract, retracting the positioning element, or both, wherein the tissue is positioned within the lumen of the tissue collector element, and (f) separating tissue positioned within the lumen of the tissue collector element from other tissue of the mammal, thereby obtaining the tissue biopsy. Step (f) can comprise using a cutting element to separate tissue positioned within the lumen of the tissue collector element from other tissue of the mammal, thereby obtaining the tissue biopsy. Step (f) can comprise using a snare element to separate tissue positioned within the lumen of the tissue collector element from other tissue of the mammal, thereby obtaining the tissue biopsy. The method can comprise removing the tissue collector element from the mammal. Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a side view of an exemplary device for obtaining biopsy material from the gastrointestinal tract of a mammal.

FIG. 2 is a view of the distal end region of a tissue collector of the device of FIG. 1.

FIG. 3 is a side view of an exemplary device for obtaining biopsy material from the gastrointestinal tract of a mammal.

FIG. 4 is a side view of an exemplary device for obtaining biopsy material from the gastrointestinal tract of a mammal.

FIG. 5 is a side view of an exemplary device for obtaining biopsy material from the gastrointestinal tract of a mammal together with mammalian gastrointestinal tissue.

FIG. 6 is a side view of an exemplary device for obtaining biopsy material from the gastrointestinal tract of a mammal.

FIG. 7 is a side view of one example of a tissue collector.

### DETAILED DESCRIPTION

This document provides methods and materials involved in obtaining biopsy material from the gastrointestinal tract of a mammal (e.g., a human). For example, this document provides devices (e.g., percutaneous biopsy devices) configured to allow a user (e.g., a surgeon) to obtain biopsy material from the gastrointestinal tract of a mammal in a minimally-invasive manner as well as methods for using biopsy devices.

In general, an outer hollow member (e.g., a catheter, needle, cannula, or tubular member) can be configured such that it can be advanced within a mammal's body to a position such that a gastrointestinal tissue sample can be obtained. In some cases, the outer hollow member can be the outer most component of a biopsy system described herein, or it can be housed completely or at least partially within another component (e.g., a sleeve or sheath). An outer hollow member can be any appropriate shape. For example an outer hollow member can have a non-round shape (e.g., an outer cross-sectional). In some cases, the lumen defined by an outer hollow member can have a non-round shape. For example, an inner surface of an outer hollow member can have a non-round cross-sectional shape (e.g., a circular shape with one or more straight or substantially straight edges).

In some cases, the outer hollow member can be configured to receive an inner tissue collector element. The inner tissue collector element can include a lumen configured to house a desired gastrointestinal tissue sample that was extracted from the gastrointestinal tract of a mammal. In some cases, an inner tissue collector element can include an extraction mechanism that can be used to remove a desired gastrointestinal tissue sample from the gastrointestinal tract of a mammal. Such an extraction mechanism can be one or more cutting blades. In some cases, a positioning element can be used to hold gastrointestinal tissue in place while a tissue collector element is advanced into the tissue during a tissue removal process. For example, a screw-like positioning element can be anchored into gastrointestinal tissue in the region where a gastrointestinal tissue sample is to be removed. Then, a tissue collector element having a distally located extraction mechanism (e.g., blades) can be inserted into the tissue in a manner such that the positioning element provides an appropriate degree of leverage for the tissue collector to advance a desired depth into the tissue. Once advanced the desired depth, the extraction mechanism can be used to free the desired gastrointestinal tissue to a point such that the tissue remains within a lumen of the tissue collector element as the tissue collector element is withdrawn from the mammal. In some cases, a system provided herein can include a clip or adhesive configured to close an opening created by the removal of the gastrointestinal tissue.

The methods and materials provided herein can be used to obtain any appropriate type of gastrointestinal tract tissue such as stomach, small bowel, and/or colon tissue. In some cases, the methods and materials provided herein can be used to obtain core samples having a diameter between about 1 mm and 10 mm (e.g., between about 2.5 mm and 10 mm, between about 5 mm and 10 mm, between about 1 mm and 7.5 mm, between about 1 mm and 5 mm, between about 2.5 mm and 7.5 mm, between about 2 mm and 6 mm, or between about 3 mm and 5 mm). In some cases, the size of the sample can be limited by the acceptable size of the wound on the abdomen and/or size of the hole in the gastrointestinal tract tissues. In some cases, the methods and materials provided herein can be used to obtain a sample of a particular selected depth (e.g., a depth selected to obtain desired tissue or tissue having a desired cell type). For example, the methods and materials provided herein can be used to obtain core samples having a depth such that the core includes serosa, muscle, submucosa, and/or mucosa. In some cases, the methods and materials provided herein can be used to obtain a tissue sample having enteric nerves, interstitial cells of Cajal (ICC), and/or immune cells.

The methods and materials provided herein can be used with or without ultrasound guidance. In some cases, the methods and materials provided herein can be used with or without endoscope illumination. For example, a system provided herein can be designed to include fiber optics. In some cases, a device or system provided herein can include one or more optical fibers. Such optical fiber can be used to determine which layer of tissue is being penetrated. For example, each tissue layer (e.g., fat, serosa, or submucosa) can be displayed as a different reflectivity that can be determined by intensity or color. The optical fibers can be used to incorporate a mechanism for conducting light from a distal tip region of a device back outside the body to a detector system capable of determining intensity changes and/or color changes. If selected monochromatic or filtered light of specified wavelengths are transmitted into the body, the reflectivity of the various components or structures of tissue can be absorbed or reflected in characteristic ways. These changes can be used to indicate when a layer or structure is initially touched, to detect contraction, and to detect movement through a layer(s).

In some cases, one or more optical fibers can be used to aid in navigation to determine when the device is positioned at the top of the layer of interest, when the layer of interest is penetrated, and/or when the segment of tissue should be removed. In some cases, an optical fiber can be penetrated into the desired tissue, in advance of a cutting tip, to determine the depth of a cut to be made. In some cases, an optical system can be used to detect blood, clear fluid such as saline, or other matter that would be found during the procedure. In some cases, sophisticated optical techniques such as optical coherence tomography can be used in combination with the methods and material provided herein.

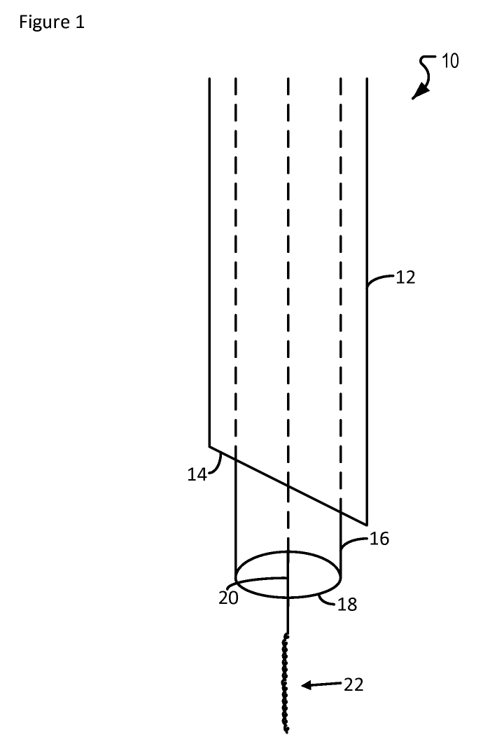

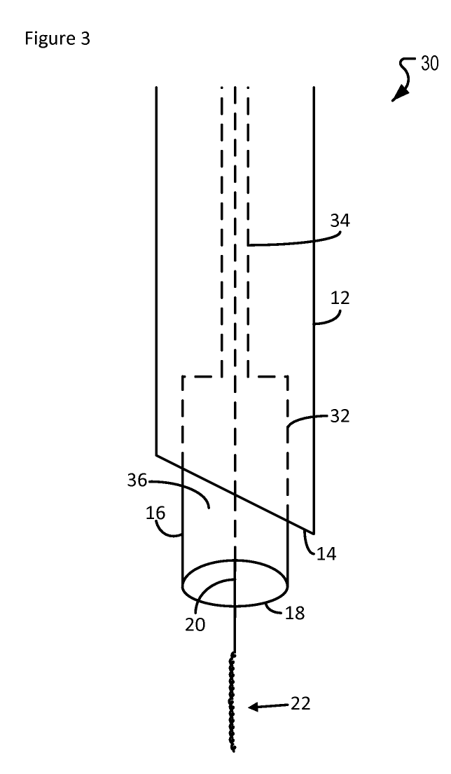

With respect to FIG. 1, device 10 can include an outer hollow member 12, a tissue collector element 16, and a positioning element 20. Outer hollow member 12 can have any appropriate diameter. For example, outer hollow member 12 can have a diameter capable of forming a slip fit with tissue collector element 16. In some cases, outer hollow member 12 can have an inner diameter between about 1 mm and about 15 mm (e.g., about 5 mm), and outer hollow member 12 can have an outer diameter between about 2 mm and about 15 mm (e.g., about 6 mm). Outer hollow member 12 can have a distal end region 14. Distal end region 14 can be pointed, sharp, curved, rounded, or blunt. In some cases, distal end region 14 can be rounded to reduce the unintentional puncturing of tissue while the outer hollow member 12 is being advanced into position within a mammal. Outer hollow member 12 can define a lumen configured to allow tissue collector element 16 to slide independently within outer hollow 12 and/or to be advance from outer hollow member 12. Tissue collector element 16 can define a lumen and can have a distal end region 18. The lumen of tissue collector element 16 can be any appropriate length. In some cases, the lumen can have the same inner diameter for the entire length of the lumen as shown in FIG. 1. In some cases, tissue collector element 16 can define a lumen that is larger at the distal end than at the proximal end. For example, as shown in FIG. 3, tissue collector element 16 can define a large lumen section 32 and a narrow lumen section 34. In some cases, the length of large lumen section 32 can be designed to match the depth of the tissue sample to be collected.

Distal end region 18 of tissue collector element 16 can be pointed, sharp, curved, rounded, or blunt. In some cases, distal end region 18 can be sharp so that desired tissue can be removed from a mammal as tissue collector element 16 is advanced into the tissue. The lumen of distal end region 18 can be configured to house a tissue sample that is removed from a mammal.

Positioning element 20 can be configured to advance independently from within outer hollow member 12 or tissue collector element 16. For example, as shown in FIG. 1, positioning element 20 can be configured to advance past distal end region 18. Positioning element 20 can be configured to advance any appropriate distance past distal end region 18, depending on the proximity of distal end region 18 to the gastrointestinal tract. In some cases, the distance positioning element 20 advanced past distal end region 18 can be used to define the depth of the tissue sample to be collected. In some cases, a handle device for positioning element 20 can include position indicators to aid in determining the distance positioning element 20 is advanced beyond distal end region 18. Positioning element 20 can include a distal end region 22. In some cases, distal end region 22 can be configured to advance into tissue to be removed. For example, distal end region 22 can have a screw-like or cork screw-like configuration that is capable of providing an appropriate degree of leverage for tissue collector element 16 to advance a desired depth into tissue to be removed. In some cases, a positioning element can be configured to be a suction device or a grasping forceps-like device. In some cases, positioning element 20 can contain optical fibers to determine the layer of tissue penetrated as described herein.

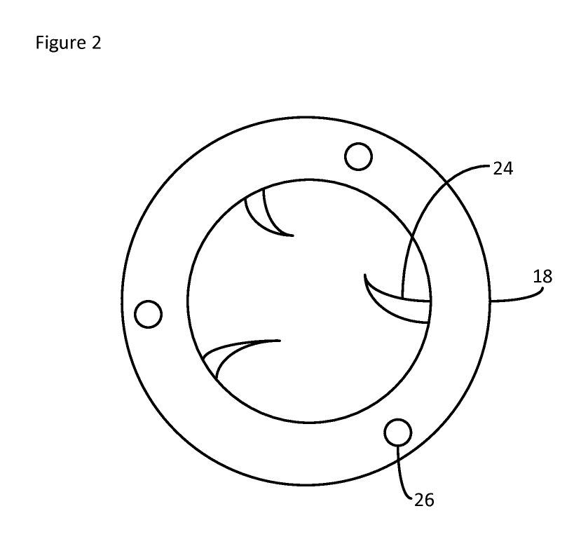

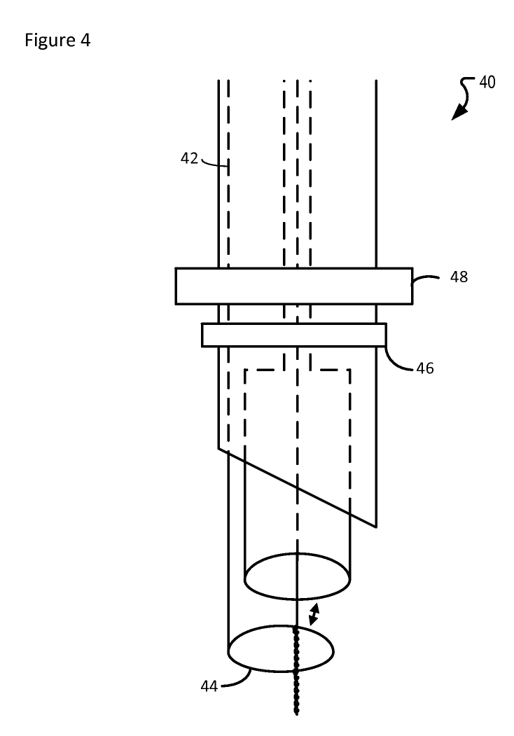

Tissue collector element 16 can be configured to have the ability to remove a tissue sample for the gastrointestinal tract of a mammal. For example, distal end region 18 of tissue collector element 16 can be sharp enough to remove a tissue sample. In some cases, tissue collector element 16 can include blades. As shown in FIG. 2, distal end region 18 of tissue collector element 16 can include blades 24 configured to cut a tissue sample from the gastrointestinal tract of a mammal. In some cases, distal end region 18 of tissue collector element 16 can include a static blade, a rotary blade (e.g., a blade capable of being turned manually or electrically), or a snare. For example, as shown in FIG. 4, distal end region 18 of tissue collector element 16 can include a snare element 42. Snare element 42 can have a proximal portion that extends within outer hollow member 12 or tissue collector element 16 and a distal portion 44. Distal portion 44 can be for a loop. In some cases, such a loop can be configured to be positioned along the distal most edge of distal end region 18 of tissue collector element 16 as shown by the arrows of FIG. 4. Such a loop can be configured to allow tissue to advance into the lumen of tissue collector element 16. Once the desired about of tissue is advanced into the lumen, snare element 42 can be actuated such that the loop frees the desired tissue to a point such that the tissue remains within the lumen of tissue collector element 16 as it is withdrawn from a mammal (e.g., a human).

Tissue collector element 16 can be configured to have one or more ports. As shown in FIG. 2, distal end region 18 of tissue collector element 16 can include ports 26. Ports 26 can be configured to allow for the delivery of substances to the location of tissue removal. For example, a sealant or adhesive material can be delivered to the opening created after tissue is removed to aid in healing.

In some cases, an outer hollow member or tissue collector element can include a bumper element configured to limit the advancement of the outer hollow member or tissue collector element into tissue. For example, as shown in FIG. 4, outer hollow member 12 can include a bumper element 46. Bumper element 46 can be a ring structure that protrudes from an outer surface of outer hollow member 12. Bumper element 46 can be a rubber-type element.

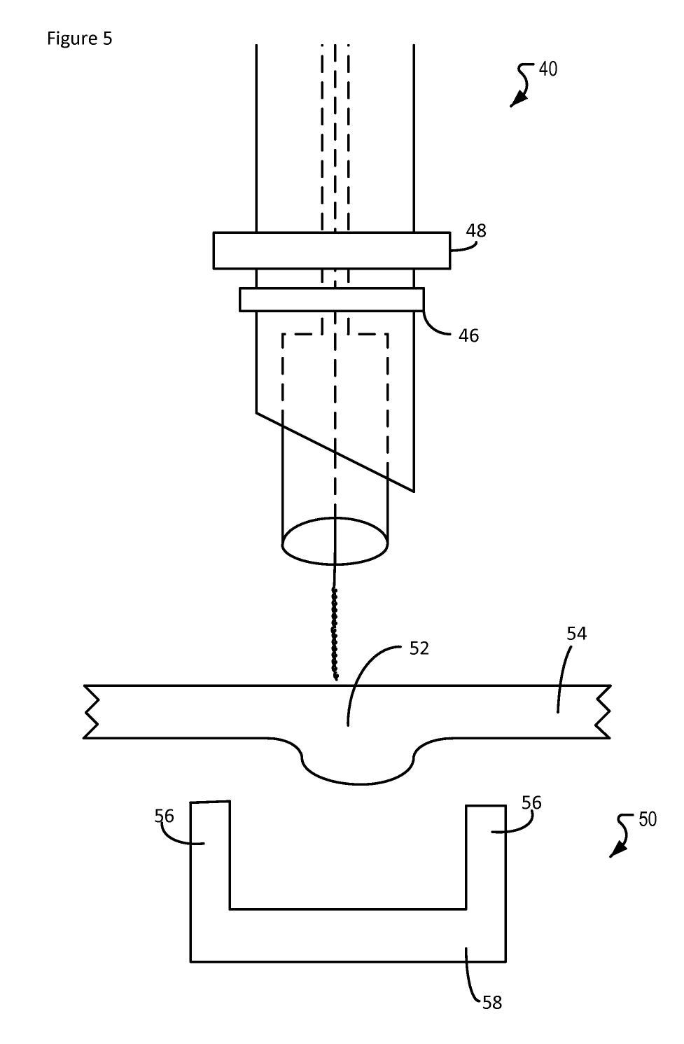

In some cases, an outer hollow member or tissue collector element can include a magnetic alignment element configured to align the outer hollow member or tissue collector element with a separate magnetic device. For example, as shown in FIG. 4, outer hollow member 12 can include a magnetic alignment element 48. Magnetic alignment element 48 can be a ring structure that protrudes from an outer surface of outer hollow member 12. Magnetic alignment element 48 can be made of magnetic material. As shown in FIG. 5, magnetic alignment element 48 can be used to align outer hollow member 12 with a separate magnetic device 50. Magnetic device 50 can include a base 58 and one or more extensions 56. Extensions 56 can be capable of magnetically associating with magnetic alignment element 48. Magnetic device 50 can be positioned in a desired location. For example, magnetic device 50 can be position to align magnetic alignment element 48 to a submucosal cushion or bleb 52 of a gastric wall 54.

In some cases, a system, outer hollow member, or tissue collector element provided herein can include a cautery element or have energy delivery capabilities. In some cases, sealants, hemostatic agents, sponge material, and/or foam material can be delivered via a lumen integral to outer hollow member or a tissue collection element. For example, gel foam, a biocompatible sponge, or a foam material such as Biomerix can be used to close or plug a created opening. In some cases, a system, outer hollow member, or tissue collector element provided herein can include a clip or other device having the ability to close an opening created in a gastric wall.

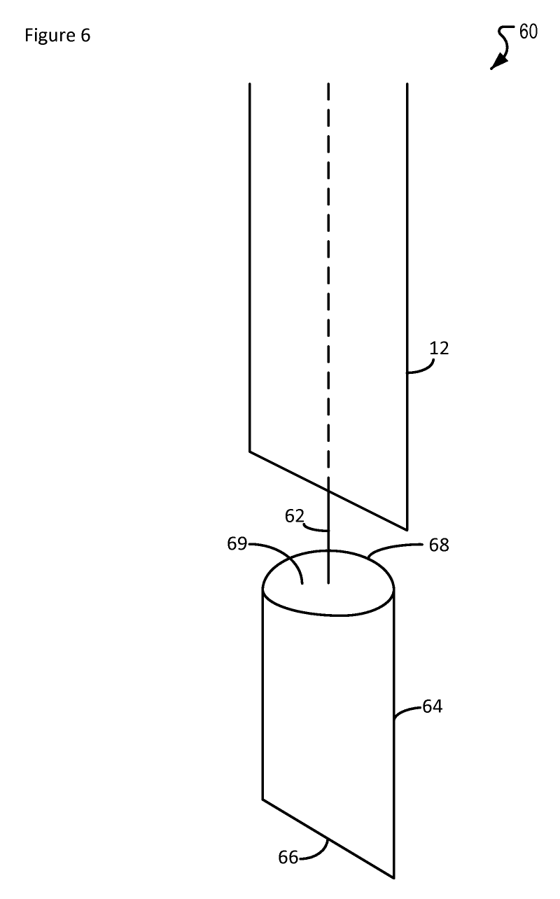

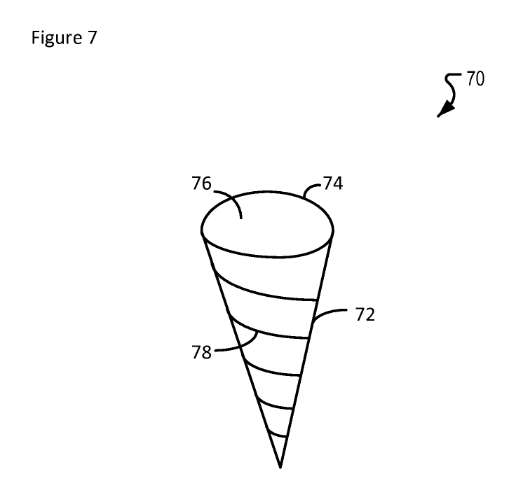

With reference to FIG. 6, device 60 can include an outer hollow member 12 and a tissue collector element 64. Outer hollow member 12 can be configured as described herein. Tissue collector element 64 can define a lumen 69 and can have a distal end region 66 and a proximal end region 68. Lumen 69 can be any appropriate length. Distal end region 66 of tissue collector element 64 can be pointed, sharp, curved, rounded, or blunt. In some cases, distal end region 66 can be sharp so that tissue collector element 64 can be advanced into desired tissue to be removed from a mammal. Proximal end region 68 can be sharp such that removal of tissue collector element 64 from tissue after being inserted results in the collection of a desired tissue sample. In some cases, proximal end region 68 can include one or more blades or other cutting elements. In some cases, tissue collector element 64 can be collapsible or expandable. For example, tissue collector element 64 can be advanced into a desired tissue in a collapsed configuration. Then, tissue collector element 64 can be converted into an expanded configuration and withdraw from the desired tissue. As tissue collector element 64 is withdrawn, a sharp edge of proximal end region 68 can cut a tissue sample free from the mammal's gastrointestinal tract. Tissue collector element 64 can have any appropriate shape. For example, as shown in FIG. 7, a tissue collector element 70 can have a cone shape. In this example, tissue collector element 70 can define lumen 76 and have an outer surface 72. Outer surface 72 can include threads 78. Threads 78 can be used to aid the insertion of tissue collector element 70 into desired tissue.

During use, an outer hollow member of a system or device provided herein (e.g., device 10) can be introduced percutaneously into a mammal (e.g., an abdominal cavity of a human). In some cases, peroral endoscopic or percutaneous methods (e.g., endoluminal methods) can be used to create a submucosal cushion prior to inserting an outer hollow member into the mammal. A tissue collector element can be introduced into the mammal by advancing the tissue collector element within the outer hollow member. Then, a positioning element can be introduced into the mammal by advancing the positioning element within the outer hollow member or the tissue collector element. The positioning element can be advanced further into tissue to be removed. At this point, the tissue collector element can be advanced into the tissue and/or the positioning element can be retracted such that the desired tissue is contained within the lumen of the tissue collector element. A cutting element or snare can be engaged to cut the desired tissue from the mammal's gastrointestinal tract. The tissue collector element containing the removed tissue can be removed as can the positioning element. A cautery, sealant, hemostatic, clip, or other appropriate agent or device can be applied to the opening to aid in its closure.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A system for obtaining a tissue biopsy from a gastrointestinal tract of a mammal, wherein said system comprises:
(a) a hollow member configured to be positioned near gastrointestinal tissue to be obtained,
(b) a tissue collection element configured to be advanced within a lumen of said hollow member, wherein said tissue collection element defines a lumen configured to house at least a portion of said tissue biopsy, and
(c) a positioning element configured to be advanced within said lumen of said tissue collection element or said lumen of said hollow member, wherein said positioning element is capable of being advanced into gastrointestinal tract tissue and capable of positioning said gastrointestinal tract tissue into said lumen of said tissue collection element upon retraction of said positioning element.

2. The system of claim 1, wherein said mammal is a human.

3. The system of claim 1, wherein said hollow member is a flexible catheter.

4. The system of claim 1, wherein said gastrointestinal tract tissue is a polyp.

5. The system of claim 1, wherein said tissue collection element comprises one or more blades.

6. The system of claim 5, wherein said tissue collection element defines one or more ports configured to deliver material from outside of the body of said mammal to the gastrointestinal tract of said mammal.

7. The system of claim 1, wherein said hollow member comprises a bumper element.

8. The system of claim 1, wherein said hollow member comprises a magnetic alignment element.

9. The system of claim 1, wherein said system comprises a snare.

10. The system of claim 1, wherein said positioning element comprises a screw-like structure.

11. A method for obtaining a tissue biopsy from a gastrointestinal tract of a mammal, wherein said method comprises:
(a) inserting a member having a distal end and a proximal end into said mammal to position said distal end of said member proximal to tissue of said gastrointestinal tract, wherein said member defines a lumen,
(b) positioning a tissue collector element within said lumen of said member, wherein said tissue collector element defines a lumen configured to house said tissue biopsy,
(c) advancing a positioning element having a distal end and a proximal end within said lumen of said member or said lumen of said tissue collector element,
(d) inserting said positioning element into tissue of said gastrointestinal tract,
(e) advancing said tissue collector element into said tissue of said gastrointestinal tract, retracting said positioning element, or both, wherein said tissue is positioned within said lumen of said tissue collector element, and
(f) separating tissue positioned within said lumen of said tissue collector element from other tissue of said mammal, thereby obtaining said tissue biopsy.

12. The method of claim 11, wherein said step (f) comprises using a cutting element to separate tissue positioned within said lumen of said tissue collector element from other tissue of said mammal, thereby obtaining said tissue biopsy.

13. The method of claim 11, wherein said step (f) comprises using a snare element to separate tissue positioned within said lumen of said tissue collector element from other tissue of said mammal, thereby obtaining said tissue biopsy.

14. The method of claim 11, wherein said method comprises removing said tissue collector element from said mammal.
